(19) United States
(12) Patent Application Publication
(10) Pub. No.: US 2023/0390310 A1
(43) Pub. Date: Dec. 7, 2023

(54) PERSONALIZED, PRECISE, CONSISTENT BOTANICAL FORMULATIONS ON DEMAND

(71) Applicant: ALTOPA, INC, Vashon, WA (US)

(72) Inventors: Nicole K. Wicker, Vashon, WA (US); L. Brent Poole, Vashon, WA (US); Erwin Stedronsky, Lakewood, CO (US)

(21) Appl. No.: 18/205,765

(22) Filed: Jun. 5, 2023

Related U.S. Application Data
(60) Provisional application No. 63/348,803, filed on Jun. 3, 2022.

(51) Int. Cl. A61K 31/00; A61K 36/185; A61P 25/04

(52) U.S. Cl. CPC A61K 31/658; A61K 36/185; A61P 25/04

(57) ABSTRACT

Formulations for pain treatment include an effective amount of liquid composition comprised of CBD, oftentimes CBG and CBN, sometimes CBC and THC, and a nominal effective amount of at least two terpenes from the group of terpenes including beta caryophyllene (CAR), α-pinene, β-pinene, limonene (LIM), linalool (LOL), myrcene (MYR), nerolidol (NRD), humulene (HUM), and terpinolene (TPN). For insomnia/nighttime pain: CBD, oftentimes CBG/CBN, sometimes THC, and nominal CAR, LEVI, LOL, MYR, NRD and TPN. For anxiety #1 (Calm): CBD, oftentimes CBG/CBN, sometimes THC, and nominal CAR, LEVI, LOL, MYR, and NRD. For anxiety #2 (Serenity), alter #1 and add α-pinene, β-pinene, HUM and TPN. For GI relief, CBD, oftentimes CBG/CBN, sometimes THC, and nominal ALL nominal CAR, α-pinene, LEVI, LOL, MYR, and NRD. Other formulations are provided for relief of: insomnia due to pain, anxiety, etc.; neurodegenerative diseases; headaches, migraines and associated nausea and vomiting; autism; and cancer and cancer treatment-related symptoms.

This non-provisional patent application is based upon and claims the benefits of provisional patent application Ser. No. 63/348,803, filed Jun. 3, 2022, now pending, the contents of which is incorporated herein by reference thereto. Additionally, the contents of U.S. Pat. No. 10,632,432, issued Apr. 28, 2020; U.S. patent application Ser. No. 17/911,309, filed Mar. 12, 2021; and U.S. patent application Ser. No. 16/652,936, filed Oct. 9, 2018 (PCT/US2018/055054), are incorporated herein by reference thereto.

### FIELD OF TECHNOLOGY

The present disclosure is in the field of botanical formulations, and more particularly, in the field of creating personalized, precise, consistent botanical formulations on demand.

### BACKGROUND

The clinical issues and problems confronting the compositions of botanical formulations that include cannabinoids such as cannabidiol (CBD), oftentimes cannabigerol (CBG) and cannabinol (CBN), sometimes cannabichromene (CBC) and tetrahydrocannabinol (THC), with various terpenes (for example, beta caryophyllene, alpha pinene, beta pinene, limonene, linalool, myrcene, nerolidol, humulene and terpinolene), include, without limitation: (a) cannabis strains have a high degree of variation in cannabinoid composition and especially in terpene profile; (b) raw flower and extracts do not contain the correct mix of components to address specific symptoms; (c) product supply is inconsistent; (d) most medical marijuana treatment centers (dispensaries) are not medically focused; and, (e) most commercial CBD products lack the potency and components needed to provide therapeutic benefits. The present inventive system and compositions solve these problems.

Terpenes and flavonoids are widely distributed in plants. Cannabinoids, found primarily in cannabis plants, can be chemically regarded as a subset of terpenes. Regarding cannabinoids, over 175 have been discovered in cannabis plants, the most common of which are THC and CBD, followed by CBG and CBN. CBD is cannabidiol, CBG is cannabigerol, CBN is cannabinol. Regarding terpenes, there are over 20,000 different compounds, and about 400 in cannabis, the most common of which are beta caryophyllene, linalool, pinene, myrcene and limonene. Regarding flavonoids, there are about 8,000 different compounds, and about 28 flavonoids in cannabis, the most common of which are cannaflavins A, B and C, orientin, quercetin, silymarin and kaempferol.

Terpenes, combined with cannabinoids, increase efficacy. Terpenes work with cannabinoids to provide an “entourage effect” of the composition in its interaction with the CB1 and CB2 receptors of the human body. For example, see Russo, Ethan B., “Taming THC,” British Jour. Of Pharmacology 2011; and Nuutinen, Tarmo, European Journal of Medicinal Chemistry 2018, European Jour. Of Medicinal Chemistry 157 (2018).

Many terpenes and cannabinoids, tested as single compounds, have been measured to have agonist, “inverse” agonist, and/or antagonist effects on these receptors. The multiplicity of actions per compound, make it almost impossible to reliably predict the physiological response to a given mixture of terpenes and cannabinoids. In the case of mixtures, the physiological response must be determined empirically and can vary from individual to individual.

The clinical issues and problems confronting the cannabinoid and terpene compositions include, without limitation: (a) cannabis has a high degree of variation in cannabinoid composition and especially in terpene profile, even for a given chemovar/strain; (b) raw flower and extracts do not contain the correct mix of components to address specific symptoms; (c) product supply is of inconsistent composition; (d) most medical marijuana treatment centers (dispensaries) are only marginally medically focused; and (e) most commercial CBD products lack the potency and components needed to provide therapeutic benefits.

Definitions and Abbreviations. The following tables provide abbreviations of some items discussed herein.

Cannabinoid Table

| | |
|---|---|
| CBC | Cannabichromene |
| CBCA | Cannabichromene acid |
| CBCV | Cannabichromevarin |
| CBD | Cannabidiol |
| CBDA | Cannabidiol acid |
| CBDV | Cannabidivarin |
| CBE | Cannabielsoin |
| CBG | Cannabigerol |
| CBGA | Cannabigerol acid |
| CBGV | Cannabigerol propyl variant |
| CBN | Cannabinol |
| CBNA | Cannabinolic acid |
| CBL | Cannabicyclol |
| CBLA | Cannabicyclolic acid |
| CBT | Cannabitriol |
| D8 THC | delta-8-tetrahydrocannabinol |
| D9 THC | delta-9-tetrahydrocannabinol |
| THC | Tetrahydrocannabinol |
| THCA | Tetrahydrocannabinolic acid |
| THCV | Tetrahydrocannabivarin |
| THCVA | Tetrahydrocannabivarin acid |

Terpene Table

| | |
|---|---|
| Alpha Bisabolol | BIS |
| Alpha Pinene | αPIN |
| Beta Caryophyllene | CAR |
| Myrcene | MYR |
| Beta Pinene | βPIN |
| Humulene | HUM |
| Limonene | LIM |
| Linalool | LOL |
| Nerolidol | NRD |
| Terpinolene | TPN |

Terpenes, isolated as pure chemical compounds, or isolated as complex mixtures from their botanical sources, may be used as botanical drug substances. Some terpenes have cannabimimetic activities. The problem is that the cannabis plant currently has innumerable strains and each different strain or chemical variety of cannabis (sometimes called a chemovar), has its own mixture of terpenes and cannabinoids. There are hundreds, if not thousands, of chemovars, each with humorous names that often allude to the kind of sensory experience a user may feel.

Further, research shows even if two products were created from the same chemovar an inconsistent mixture of terpenes and cannabinoids may result. Variations in temperature, growing medium, nutrients, and sunlight alter cannabinoid and terpene compositions in a given chemovar.

### SUMMARY

According to an aspect of the present disclosure, a system is provided, including a device for creating personalized, precise, and consistent botanical formulations.

According to an aspect of the present disclosure, a method is provided, including creating personalized, precise, and consistent botanical formulations.

Formulations for the treatment or amelioration of pain include an effective amount of liquid composition comprised of cannabidiol (CBD), oftentimes cannabigerol (CBG) and cannabinol (CBN), sometimes cannabichromene (CBC) and tetrahydrocannabinol (THC), and a nominal effective amount of at least two terpenes from the group of terpenes including beta caryophyllene, alpha pinene, beta pinene, limonene, linalool, myrcene, nerolidol, humulene, and terpinolene to a patient suffering from pain.

Inventive compositions for insomnia and nighttime pain comprise a liquid composition comprised of cannabidiol (CBD), oftentimes cannabigerol (CBG) and cannabinol (CBN), and sometimes tetrahydrocannabinol (THC), and a nominal effective amount of a terpene, from the group of terpenes, including myrcene, nerolidol, beta caryophyllene, terpinolene, linalool, and limonene to a patient suffering from insomnia.

Inventive compositions for anxiety #1 (Calm) (defined later) include treatment of anxiety and post-traumatic stress disease (PTSD), comprising a liquid composition comprised of cannabidiol (CBD), oftentimes cannabigerol (CBG) and cannabinol (CBN), and sometimes tetrahydrocannabinol (THC), and an effective amount of at least one terpene from the group of terpenes including beta caryophyllene, limonene, linalool, myrcene, and nerolidol, to a patient suffering from anxiety.

Inventive compositions for anxiety #2 (Serenity) (defined later) include a liquid composition comprised of cannabidiol (CBD), at least one of cannabigerol (CBG), cannabinol (CBN), and tetrahydrocannabinol (THC), and an effective amount of at least one terpene from the group of terpenes including beta caryophyllene, limonene, linalool, alpha pinene, beta pinene, myrcene, nerolidol, humulene, terpinolene, linalool, myrcene, and nerolidol, to a patient suffering from anxiety.

Inventive compositions for irritable bowel syndrome include a liquid composition comprised of cannabidiol (CBD), oftentimes at least one of cannabigerol (CBG), cannabinol (CBN), and tetrahydrocannabinol (THC), and an effective amount of at least one terpene from the group of terpenes including beta caryophyllene, alpha pinene, limonene, linalool, myrcene, and nerolidol, to a patient suffering from irritable bowel syndrome.

### BRIEF DESCRIPTION OF THE DRAWINGS

Further objects and advantages of the present invention can be found in the accompanying drawings, when taken in conjunction with the specification, which follow.

FIG. 1 shows pain formulations recommended to patients based on their medical condition and represent the patient's first or BASE formulation (TF indicates that the formulation does not include THC, D indicates that the formulation includes THC).

FIGS. 2 and 3 show Oblend® Treatment Family (Oblend Tx) Calm formulations and Oblend Tx Serenity formulations. These tables include base formulations (BASE) for subjects suffering from anxiety, depression, and PTSD.

FIG. 4 shows Oblend® Tx Wisdom Base Formulations to relieve symptoms associated with gastrointestinal disorders, such as IBS and others.

FIG. 5 shows Oblend® Tx Rest BASE formulations which provide relief to subjects suffering from insomnia and difficulty sleeping from pain, anxiety, and other similar medical conditions.

FIG. 6 shows Oblend® Tx BASE Formulations called “Reagan” which provides relief for patients suffering from neurodegenerative diseases, including memory loss, Alzheimer's disease, Parkinson's disease, ALS, and Huntington's disease.

FIG. 7 shows Oblend® Tx BASE Formulations called “Relief MG” which provide relief for subjects suffering from headaches and migraines, including relief from associated migraine symptoms of nausea and vomiting.

FIG. 8 shows Oblend® Tx BASE Formulations for Autism called “Halo.”

FIG. 9 shows Oblend® Tx BASE Formulations called “Armstrong” providing relief to subjects suffering from cancer and cancer treatment-related symptoms.

FIG. 10 is the symptom-specific Pain Management Table;

FIG. 11 is the symptom-specific Insomnia Table;

FIG. 12 is the symptom-specific Cancer Treatment Support Table;

FIG. 13 is the symptom-specific Anxiety, Depression, PTSD Management Table;

FIG. 14 is the symptom-specific Chronic Obstructive Pulmonary Disease (COPD) Table; and

FIG. 15 is the symptom-specific Irritable Bowel Syndrome Table. These are symptom-specific tables which summarize the pharmaceutical compositions where two or more patients, grouped by specific composition have reported moderate to substantial symptom improvement.

### DETAILED DESCRIPTION

The present disclosure includes systems and methods for creating personalized, precise, consistent botanical formulations on demand in the field of natural medicine. This can involve a specialized device to facilitate delivery of the botanical formulation as treatment.

According to one implementation, this includes replaceable canisters. Like an inkjet printer, these replaceable, EEPROM-encoded canisters can be filled with quality-controlled ingredients.

According to one implementation, ingredients include botanical extracts, including cannabinoids, isolated terpenoids, therapeutic essential oils & flavonoids.

According to one implementation, botanical formulations can be created and customized with pharma-grade precision. According to one implementation, the technology of the present disclosure includes microfluidic technology which permits personalization and pharma-grade precision. According to one implementation, ingredients can be dispensed in amounts of <1 mg.

According to one implementation, botanical formulations can include no THC. Then THC can be added to subsequent formulations in amounts ranging from 1-10 mg/ml.

According to another aspect of the present disclosure, the specialized device can operate with no additional heat to facilitate an ambient temperature permitting the addition of acidic ingredients, such as THCA and CBDA. Additionally, other implementations of the specialized device can include positive displacement pumps for dispensing THCA and CBDA or other higher viscosity ingredients at room temperature. Additionally, other implementations of the specialized device can operate at ambient temperatures.

According to another implementation of the present disclosure, a base formulation (BASE) can be utilized as a substitute for D8 THC and/or D9 THC.

According to another implementation of the present disclosure, formulations within the present disclosure can be used to treat addicts (such as, opiate addicts) by using the specialized device to decrease opiates while introducing or increasing botanical formulations as a replacement. For example, these opiates could be dispensed from the specialized device, or separately. According to the illustrative implementation, the patient would not know how much of each product they are getting (similar to, or as part of, a clinical trial). Replacement formulation could include CBD, beta Caryophyllene, myrcene, and may or may not include THC and other ingredients. It should be noted that this formulation could be used for treating other addicts as well.

According to one implementation, the present disclosure includes a user and healthcare (HC) provider platform to guide administration of the formulations. This platform can include HC provider formulations, permit searching by symptom, use, ratings, ingredients, and/or ingredient. The platform includes product customization, and allows a HCP (healthcare provider) user to find, replicate and customize effective treatments, adjust ingredient ratios and potency, create personalized dosing, and/or save formulations to provider/patient E.H.R. profiles. The platform includes modules: HCP portal, and patient portal, formulator processor toolkit, and a fulfillment suite.

According to one implementation, the present disclosure includes assisting HCP with prescribing, including assisting with: (1) Which formulation to recommend for what medical condition; (2) feedback whether the formulation helped the patient; and/or (3) alternate formulations of possibly improved efficacy for this patient.

According to one implementation, the present disclosure includes assisting HCP in selecting and recommending proven botanical formulations for patients; adjusting formulation based on patient feedback; accessing patient's history of formulations; receiving monthly patient reports; and/or improving quality of care. This can involve, as an illustrative example, steps (1) a patient office visit (or telemedicine), (2) selecting and recommending a formulation from the platform, (3) patient approval and payment, (4) creation and shipment of the formulation to the patient, and, (5) based on patient feedback, care providing adjusting formulation (if necessary)

The Oblend® machine, disclosed in U.S. Pat. No. 10,632,432, issued Apr. 28, 2020; U.S. patent application Ser. No. 17/911,309, filed Mar. 12, 2021; and U.S. patent application Ser. No. 16/652,936, filed Oct. 9, 2018 (PCT/US2018/055054), creates personalized botanical formulations with pharma-grade precision. The Oblend® platform and methodology described herein gives care providers the ability to find efficacious formulation and customize them based on patient feedback. These quality-controlled ingredients include: high purity isolates of cannabinoids such as THC, CBD, CBN and CBG and high purity isolates of terpenes such as CAR, LEVI, LOL, αPIN, and/or (SPIN.

Terpenes, isolated as pure chemical compounds, or isolated as complex mixtures from their botanical sources, may be used as botanical drug substances. The present invention establishes that there are health benefits when cannabinoids, such as THC and CBD, are synergistically combined with terpenes, such as beta caryophyllene and limonene. Some terpenes have cannabimimetic activities. The studies, discussed herein, show that the terpenes, listed in the Terpene Table above, when combined with cannabinoids, produce an entourage effect that modulates the effects of the cannabinoids in a way that results in unique and improved patient outcomes.

Because the cannabis plant has innumerable strains and each different strain or chemical variety of cannabis (a chemovar) has its own mixture of terpenes and cannabinoids, the studies herein establish that certain combinations of terpenes and cannabinoids can provide relief to some disease conditions. These problems are solved by the present invention which studied and established the use of certain cannabinoids and terpenes to create very precise, consistent cannabinoid formulations. The formulations used in this study were precisely produced by the OBLEND® machine described in U.S. Pat. No. 10,632,432, issued Apr. 28, 2020; U.S. patent application Ser. No. 17/911,309, filed Mar. 12, 2021; and U.S. patent application Ser. No. 16/652,936, filed Oct. 9, 2018 (PCT/US2018/055054), the contents of which are incorporated herein by reference thereto. Using the OBLEND method, each cannabinoid and terpene can be dispensed in very precise, consistent amounts in amounts ranging from less than 1 mg to the amounts set forth in the present invention.

The present formulations permit dosage titration protocols which, over time, improves the patient's condition. In some cases, patients may take higher doses during the first few months to reduce an inflammation to experience immediate symptom relief. Once their symptoms are alleviated, the healthcare provider (HCP) lowers (down titrates) the dosage amount to prevent the medical condition from reoccurring. In other cases, patients take lower doses during the first few months and slowly increase (up titrates) the daily dosage amount. Once the patient's medical condition is under control, the HCP lowers the dose. Titration, as described herein to achieve personalized medicine for the patient, is a way to limit potential side effects by taking time to see how the patient's body reacts to the formulated drug. In up-titration the medication is started at a low dose. Every couple of weeks, the dose is raised until the maximum effective dose (“target dose”) has been achieved or side effects occur. Drug titration protocols are a way for HCPs to personalize medication doses so that patients can obtain the intended benefits of the treatment of their disease while minimizing side effects.

Increased tolerance is another consideration resolved by the present invention. A patient's body may build up tolerance to cannabinoids. In dosing, increased tolerance to THC is common. The formulations set forth herein take this issue into account by the use of titration protocols and personalization of the formulation.

In pharmacology, there is an ongoing tension between botanical versus synthetic drugs. Although over 50% of pharma industrial products are plant-derived, after medicinal properties of the plant are discovered, many companies mass produce commercial products by synthetically producing the drug, mainly to maintain consistently. Studies suggest that many patients suffer from the side-effects of synthetic materials in prescribed drugs. The FDA has recently signaled its recognition that botanical drugs may be provide safer solutions than synthetic versions and has created a pathway for clearance and approval.

One of the several beneficial aspects of the present formulation is the use of botanical components without sacrificing consistency. Studies and formulation described herein benefit from the facts that; (A) botanical drugs are multi-faceted (one base formulation can target multiple medical conditions); (B) ingredients in botanical drugs work well together with less to no adverse drug interactions in contrast to targeted synthetic drugs; and (C) until recently, the FDA has only approved two (of 800) botanical drugs and recently approved the first botanical drug that included a cannabinoid, CBD (Epidiolex®). The present studies discussed herein establish that more than one ingredient is necessary to activate the cannabinoid-terpene entourage effect, that is, to open or block receptors, modulate the identified cannabinoid (CBC, CBD, CBG, CBN and THC), and target specific medical conditions.

The following tables include formulations recommended to patients based on their medical condition(s). In some embodiments, patients reported a reduction in symptoms with a formulation that included CBD. These formulations represent the patient's first formulation or “Base Formulation” (BASE). Shortly before the patients receives the formulation, patient data is collected from a variety of sources including emails, texts, phone calls, and surveys, including patient demographics, current medications, primary and secondary medical conditions, as well as baseline information. After the patient uses the formulation, feedback is collected from the patient, including efficacy, dosing protocol, side effects, and changes to current medications, if any. If, based on the patient's feedback, the formulation is providing the desired relief, then the formulation is not adjusted, and the patient can order the same formulation. If the patient is not receiving the desired outcome, then, based on the patient's feedback, the patient's 2nd formulation may be (a) a different formulation within the same Oblend Treatment Family (“Oblend Tx”), (b) a formulation from a different Oblend Tx, or (c) a personalized formulation not included in the tables below. This process continues until the patient finds the optimal formulation that addresses the patient's unique needs.

In connection with all the formulations set forth in the Tables, the following general considerations should be taken into account. (A) If a formulation did not include THC (or only included the cannabinoid CBD), typically there was a need to include the terpene beta Caryophyllene (given its impact to the CB2 receptors) to achieve positive patient outcomes. (B) If the BASE of subsequent formulations did NOT include THC, and thereafter THC was added, only a small amount of THC (as low as 1 mg/ml) was typically needed to produce or increase symptom relief. THC was added to the formulation. (C) When a small amount of THC (1-5 mg/ml) was added to a formulation that only included one cannabinoid (CBD), then the amount of CBD may be reduced significantly to produce the same outcome or to produce increased patient outcomes, depending on the patients' medical condition. For example, 108 mgs of CBD and no THC. If 2 mgs of THC was added, then CBD can be reduced to 22 mgs (10:1 ratio) in order to produce the same effectiveness in symptom reduction. (D) For example, based on the studies, a pain patient may receive about −100 mg/ml of CBD (and would usually take 0.25-1 ml dropper 2-3× per day). If 1-2 mg/ml of THC was added to the formulation, then the CBD may be reduced from 100 mgs to 10-20 mgs/ml (a 10:1 ratio of CBD to THC) and the patient then may experience the same pain reduction outcome or an increased reduction in pain symptom. (E) If certain cannabioniods that bind to the CB2 receptor, such as THC and CBG, are not included in the formulation (that is, CBD was the only cannabinoid included in the formulation), then, based on the studies, patients received a significant improvement in symptom relief if beta Caryophyllene was included in the formulation, perhaps due to beta Caryophyllene's binding properties to the CB2 receptor. (F) Adding or increasing an individual's cannabinoids such as by adding THC, CBG, or CBC, or a combination thereof, may produce better relief outcomes. (G) Increasing the amount of an individual terpene or a combination thereof may also increase patient relief outcomes. Likewise, in some cases, decreasing cannabinoid(s) produced better patient relief outcomes.

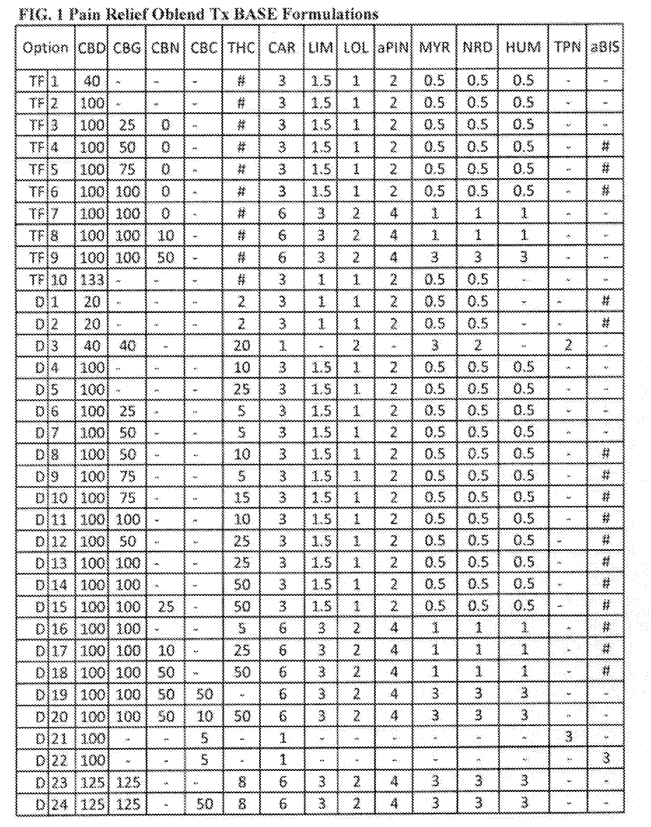

In connection to all the formulations in FIGS. 1-9 (the BASE formulations), the following is applicable. (A) The Figures show the OBLEND® Treatment Family (Oblend Tx) used by HCP to recommend botanical formulations to their patients based on the patient's presented medical condition. (B) Sometime, the HPC will start with a BASE formulation of TF 1 or D1. Then, if the patient did not experience the desired symptom relief, then the HCP will select another formulation from the designated Oblend Tx BASE. Other times, the patient's formulation will not be one from Oblend Tx BASE tables. Instead, the patient's formulation will be personalized for that patient based on the patient's unique needs. (C) For each cannabinoid and terpene listed in the following columns, the amounts represent mgs/1 ml. (D) Based on the known therapeutic properties of THCA, CBDA, THCV and CBDV, adding one or a combination of these cannabinoids can be incorporated into all formulations set forth herein. (E) Based on the known therapeutic properties of terpenes Ocimene, THCA, CBDA, THCV and CBDV, adding one or a combination of these cannabinoids can be incorporated into all formulations set forth herein. (F) TF indicates that the formulation does not include THC, D indicates that the formulation includes THC. (G) All Tx BASE formulations in Tables 1-9, if the formulation is a D formulation, there are added CBC components. (H) Further, THCa, CBDa, THCv, CBDv, may be included in the formulations.

FIG. 1 Table shows pain formulations recommended to patients based on their medical condition and represents the patient's first or BASE formulation (TF indicates that the formulation does not include THC, D indicates that the formulation includes THC). FIG. 1 Table shows Oblend®'s Relief Treatment Family (Oblend Tx). Relief includes Base Formulations used in connection with more than one patient suffering from pain and/or inflammatory diseases including neuropathic, and nociceptive pain caused by a range of injuries, arthritis, fibromyalgia, fibrosis, shingles, diabetes, and Lyme disease. Some patients suffering from pain were also suffering from COPD. Based on the studies, such patients had significantly better outcomes when the amount of terpenes were increased. For example, in the Table FIG. 1, Base Formulation TF8 has twice as much terpenes as TF7. Based on the studies, when the terpenes such as alpha-pinene and beta caryophyllene were increased, patients suffering from COPD reported significantly improved outcomes. If a patient was experience pain resulting from an inflammatory condition, such as COPD, then it was not necessary to include THC in the patient's formulations. However, if the patient was suffering from a type of pain such as nociceptive or neuropathic, the use of THC was beneficial. Increasing alpha pinene will be beneficial for patients with COPD.

In some cases, patients reported a reduction in symptoms with a formulation that included CBD, LOL, LIM, NRD, MYR, CAR, αPIN. If a patient did not experience symptom relief, then HCP adjustments were made to the formulation, included adding HUM, TPN, THC, CBG, CBC, or a combination thereof. In addition, even when a patient was reporting good symptom relief, patient dosages were titrated up, including both dosing frequency and dose size, to determine whether better relief could be achieved. At some point the patient would not experience additional symptom relief indicating that the patient's saturation level had been reached. Thereafter, the HCP would revert the formulation back to the previous dosage and constituent amounts. Treatment then continues forward at that reverted level. In most cases, the saturation level and in many cases, the most effective formulation, varied from patient to patient.

Research has shown that beta Caryophyllene is the only terpene that acts like a cannabionioid in that it activates the CB2 and the nuclear receptors and favorably modulates numerous signaling pathways within the endocannabinoid system. For example, the CB2 receptors regulate inflammation. This indicates that if a patient has inflammatory conditions and a formulation does not include ingredients to activate the CB2 receptors, then adding beta Caryophyllene in small amounts ranging from 0.5-6 mgs/ml increased patient outcomes.

CBG was also added to the BASE formulation in which case, some patients reported a reduction in symptoms. For patients who did not experience symptom relief, CBC was added to the BASE formulation and in some cases, patients reported a reduction in symptoms. Relief in Table FIG. 1 has been proven to be effective for reducing pain. If a patient was suffering from inflammatory pain, then a THC-free formulation often produced positive patient outcomes. For neuropathic pain, a small amount of THC generally resulted in improved pain relief.

Based on patient outcomes for both pain and anxiety, the BASE formulations only started with a CBD. Upon adding CBG and/or CBN, favorable patient outcomes increased. Also, for pain patients, if they did not respond to formulations that included CBD and CBG, then CBC was added, and favorable patient outcomes improved dramatically.

Based on the wide range of potential therapeutic benefits of cannabinoids, terpenes, and flavonoids, it is possible that a combination of cannabinoids and/or terpenes and/or flavonoids help alleviate symptoms or treat more than one medical conditions/ailments. For most cannabinoids and terpenes, there are safety limits on each individual ingredient and the total ingredients in the formulations (that is, the total terpenes in the formulation) relative to the total active ingredients and also relative to the total volume of formulation. For example, total amount of terpenes in relation to total active ingredients can be range anywhere between 1%-15%. This indicates that if the amount of active ingredients are increases by 2×(that is, the total cannabinoids increase from 100 mgs/ml to 200 mg/ml), then each terpene can also increase by 2× or a portion thereof.

Relief has proven to be equally effective for COPD and other inflammation-related respiratory issues due to the pinene and 48 THC (effective expectorants & bronchodilators), linalool (proven to reduce lung inflammation), and other anti-inflammatory terpenes (beta caryophyllene, myrcene, humulene, limonene, and nerolidol). If a patient is suffering from inflammatory pain, then a THC-free formulation will work. For other types of pain, such as neuropathic or nociceptive pain, a small amount of THC will result in better patient outcomes.

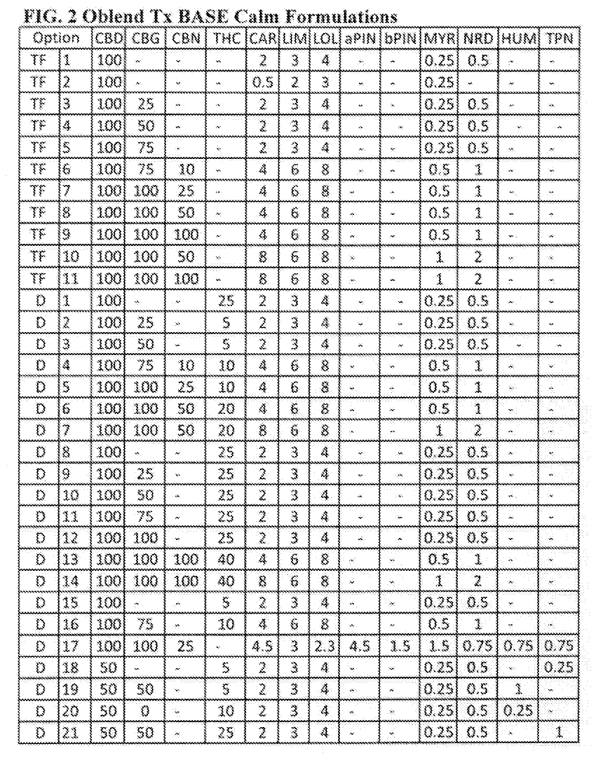

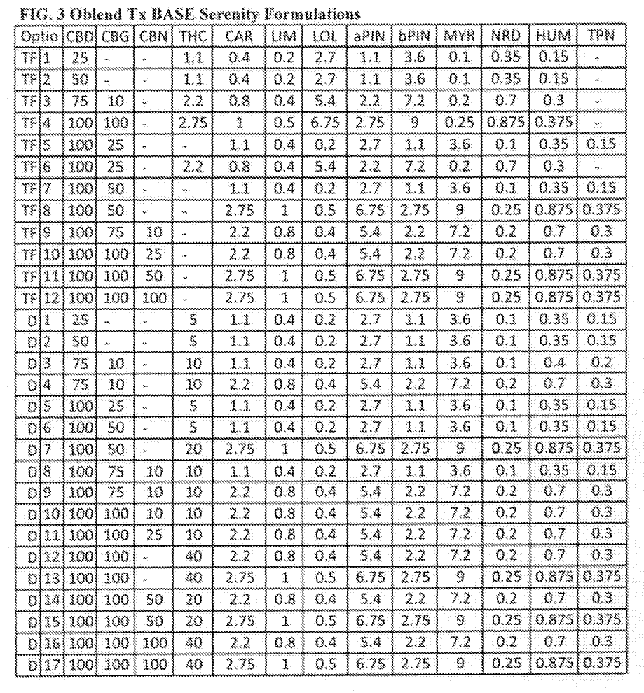

FIGS. 2 and 3 show Oblend® Treatment Family (Oblend Tx) Calm formulations and Oblend Tx Serenity formulations. These tables include base formulations (BASE) for subjects suffering from anxiety, depression, and PTSD. The BASE Formulation is selected from Calm or Serenity Tables based on the subject's symptoms.

Some patients with anxiety are also depressed and have difficulty with motivation or fear of participating in activities that may cause anxiety. Stated otherwise, some patients with anxiety suffer from social phobia involving high levels of anxiety, fear, and avoidance of social situations or places that might cause them to panic, feel trapped, helpless or embarrassed, a sense of dread, impeding danger, or doom, increased isolation, a lack of motivation, self-consciousness and concern about being judged or viewed negatively by others, paranoia, feeling weak, irritable, or tired. Based on reported patient outcomes, patients with the forementioned anxiety symptoms reported better outcomes from a Calm Tx Base Formulation versus a Serenity Tx Base Formulation. It is likely that patients with symptoms of anxiety benefit from the Calm formulations terpene profile which includes significant amounts of limonene and linalool, powerful anti-anxiety, and anti-depressants.

If the patient did not respond well to Calm, or, in addition to anxiety, has symptoms of depression, then a formulation from Serenity BASE Tx is typically recommend. Serenity has low amounts of limonene and linalool and instead, a high concentration of myrcene (a sedative) and alpha and beta pinene (anti-anxiety and anti-depressants). If the patient needs further formulation adjustments, typically the terpene profile is adjusted.

Based on patient outcomes for both pain and anxiety, initial formulations start with only CBD. Thereafter, the studies added CBG and/or CBN to the formulations. The result was that patients reported higher levels of relief from symptoms. Also, for pain patients, if they did not respond to formulations that included CBD and CBG, then CBC was added, and patient outcomes dramatically improved.

Studies show that symptoms of anxiety, depression, and PTSD are unique to each patient. Some patients respond well to LIM and LOL while others respond better to more sedative terpenes like MYR and TPN. In most cases, patients with anxiety respond well to Calm Tx formulations. However, some patients with anxiety do not respond well to LEVI, a terpene included in Calm formulations. For those patients, better outcomes were reported with the Serenity Base formulations. If a patient suffering from anxiety initially began treatment with the Calm Base and experienced some symptom relief, then, this patient continued taking Calm formulations from Calm or a personalized formulation with a similar terpene profile as Calm. However, if the patient suffering from anxiety began with the Calm Base formulation and did not experience the desired or predicted relief outcome, then the patient was transitioned to the Serenity Base formulation.

Some patients with anxiety feel tense and unable to relax. These patients may also experience racing thoughts, increased respiration rate, increased heartrate, panic attacks, hyperventilation, sweating, trembling, trouble sleeping, and difficulty concentrating.

Patients suffering from this kind of heightened anxiety reported improved symptoms from one of the Serenity terpene profiles (Serenity BASE), versus the Calm terpene profile (Calm BASE). The Serenity BASE terpene profile includes a higher concentration of MYR along with smaller concentration of TPN and NRD. This Serenity BASE terpene profile also has a high content of αPIN and βPIN in comparison to the Calm BASE. Serenity BASE also has nerolidol compared to Clam BASE.

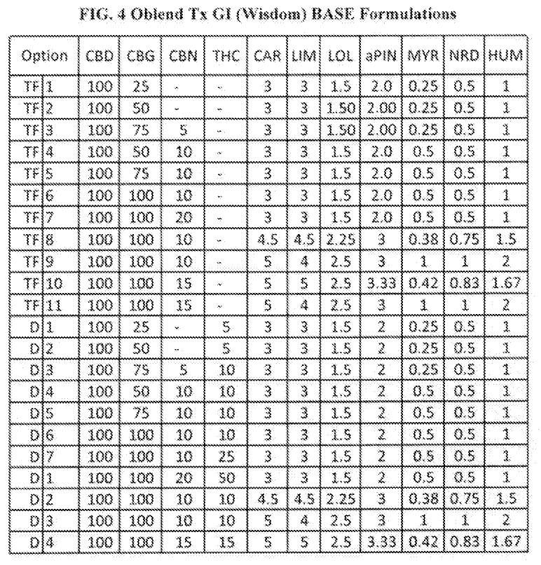

FIG. 4 Table shows Oblend® Tx Wisdom Base Formulations. Studies have shown that the Wisdom BASE formulations relieve symptoms associated with gastrointestinal disorders, such as IBS and others. Based upon patient reported feedback, the Wisdom Base formulations reduce the following symptoms: bloating, cramps, hot flashes, diarrhea, and constipation, all resulting in improved digestive interactions. In addition, it was observed that adding CBD in amounts ranging from 15-100 mg/mL improved symptom relief. Patients also reported that Wisdom BASE formulations reduced menstrual and menopausal symptoms including cramps and hot flashes.

FIG. 4 shows formulation for relief of gastrointestinal (GI) and digestive issues. The Wisdom BASE formulation relieve conditions associated with IBS, IBD, Crohn's disease, ulcerative colitis GERD disease, chronic reactive gastritis menstrual and menopausal problems. The Wisdom formulations were also created for patients with digestive issues including chronic reactive gastritis, inflammatory bowel disease and celiac disease. CBG's antibacterial and anti-inflammatory properties improves gut health and effective for a variety of digestive issues. Because they have anti-inflammatory and analgesic properties, CBD and CBG will help alleviate intestinal inflammation and any associated pain or discomfort through their analgesic properties. Other reported symptom relief includes reduced bloating, cramps during periods, hot flashes, diarrhea, and constipation. Wisdom formulations also include beta caryophyllene (which protects against gastric issues), nerolidol (used to treat gastric ulcers), and limonene (used to treat GERD). Patient expectations include some relief from inflammation resulting from gastric diseases such as IBS & IBD which conditions may have been building over many years. Patients report symptom relief with their first Wisdom formulation; however, it may take several months before the inflammation is completely significantly reduced or removed or entirely eliminated. HCPs should advise the patient that long term use of Wisdom formulations may entirely eliminate inflammation. In addition, patients may need to take higher doses of the Wisdom formulations for the first few months. Thereafter, lower doses of critical components may be provided in the patient's customized Wisdom formulation once the patient's medical condition is under control.

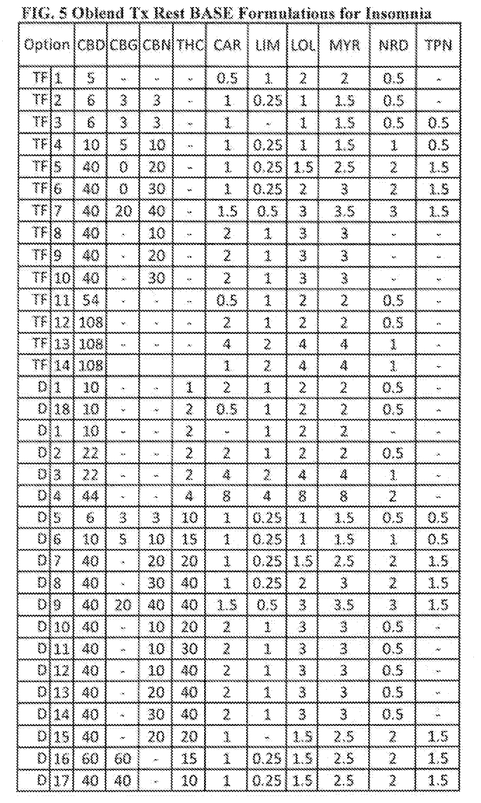

FIG. 5 Table shows Oblend® Tx Rest BASE formulations. The Rest BASE formulas provide relief to subjects suffering from insomnia and difficulty sleeping from pain, anxiety, and other similar medical conditions. Studies show that for patients suffering from a sleep disorder (e.g., hard to fall asleep), a Wisdom BASE formulation that included CBD, and sometimes CBG and CBN, plus myrcene, terpinolene, linalool, and nerolidol was not effective. However, when a small amount of THC was added to the Wisdom BASE formulation, patients were able to fall asleep.

For patients that experienced difficulties with staying asleep or waking up too early and not able to get back to sleep, studies show that a Wisdom BASE formulation without THC was effective in helping patients sleep longer or sleep without waking up throughout the night. Further observations showed that increasing the myrcene concentration by 0.25-3.5 mg/mL, and sometimes adding terpinolene and nerolidol by the same amounts, increased the duration and quality of sleep.

Conversely, if a patient woke up groggy or slept longer than desired, studies show that decreasing the amount of myrcene and sometimes decreasing the amount of linalool, terpinolene and nerolidol in dosage amounts ranging from 1.25-2 mg/mL, would alleviate these symptoms.

Most subjects that have difficulty falling asleep have increased patient outcomes when their formulation includes THC. However, if the subject has no issues with falling asleep but wakes up throughout the night, then a THC-free formulation typically produces positive patient outcomes. In several instances, the patient sleeps well but wakes up groggy. In other instances, patients sleep well, but wake up earlier than desired. In these circumstances, certain terpenes are adjusted in the formulations, such as myrcene and terpinolene, to wake up feeling fresh or to help patients sleep longer. Based on studies, increasing the myrcene, linalool and sometimes terpinolene in amounts ranging from 0.25-3 mg/ml improves sleep, including inability to stay asleep throughout the night or waking up too early. In addition, based on studies, adding a small amount of THC (0.25-10 mg/ml) to a formulation improves sleep.

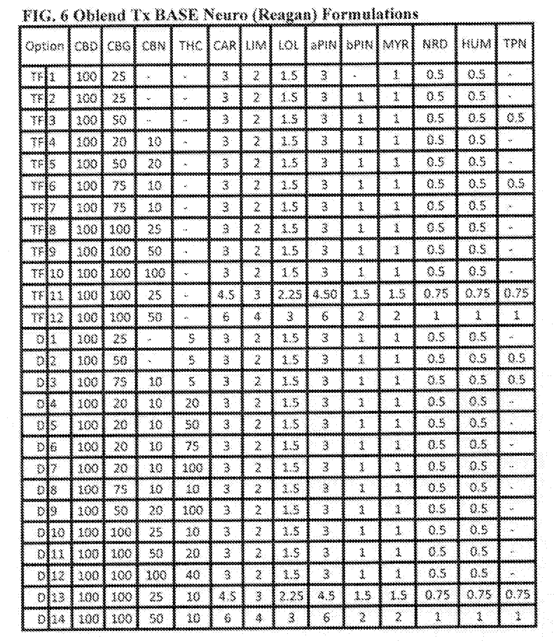

FIG. 6 Table shows Oblend® Tx BASE Formulations called “Reagan” FIG. 1.01F, Oblend Tx Reagan BASE Formulation Table provides relief for patients suffering from neurodegenerative diseases, including memory loss, Alzheimer's disease, Parkinson's disease, ALS, and Huntington's disease. In addition, studies show that this combination of cannabinoids and terpenoids provides relief to patients as a neuroprotector against these neurodegenerative diseases. Based on patient reported feedback, Reagan BASE formulations resulted in a decrease in tremors for patients with Parkinson's Disease. It also helped patients with memory loss and agitation. In addition, several patients submitted their bloodwork results which indicated a decrease in blood pressure.

The Reagan BASE formulations are recommended by HCP for patients suffering from neurodegenerative diseases, memory loss, Alzheimer's Disease, Parkinsons Disease, ALS, and Huntington's Disease. The Reagan BASE was also recommended by HCP as a neuroprotector against neurodegenerative diseases. Reagan Oblend Tx BASE includes a rainbow or combination of terpenes including: (a) alpha Pinene (b) beta Caryophyllene, (c) Humulene, (d) Limonene, and (f) Nerolidol. Based on studies, this terpene profile, in combination with the cannabinoid profiles, (1) reduced the number of tremors in patients with Parkinson's Disease, (2) decreased agitation and memory loss in Alzheimer's patients, and (3) decreased pain and anxiety associated with neurodegenerative diseases such as Alzheimer's and Parkinson's Disease.

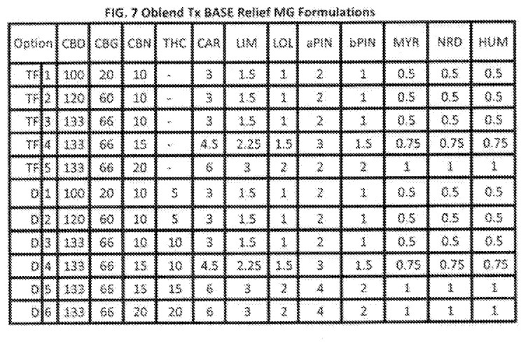

FIG. 7 Table shows Oblend® Tx BASE Formulations called “Relief MG.” The Oblend Tx BASE Relief MG formulations provide relief for subjects suffering from headaches and migraines, including relief from associated migraine symptoms of nausea and vomiting.

The Relief MG BASE formulations are recommended by HCP for patients suffering from pain associated with migraines. Based on studies, patients experience symptom relief from at least one of the Relief MG BASE formulations. Relief MG is a slight variation of Relief Oblend Tx BASE but is adjusted slightly to specifically target migraines. Patients reported nighttime pm favorable outcomes. Also patients reported migraines pain relief in the daytime am.

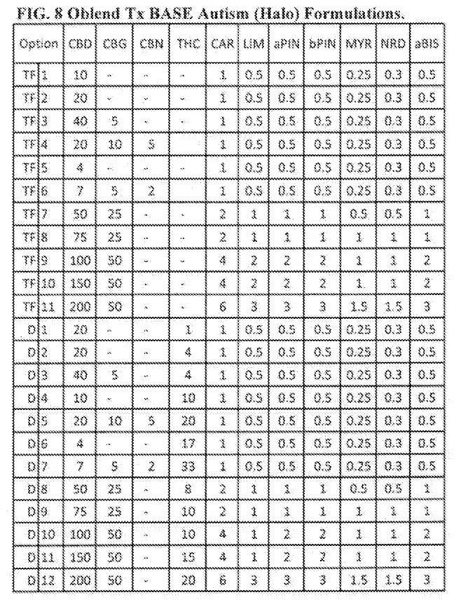

FIG. 8 shows Oblend® Tx BASE Formulations for Autism called “Halo.” Autism is very individualized. Therefore, the treatment must be individualized for each patient. This treatment plan is based on 3 existing products that have shown high efficacy for patients with autism: a 20:1 ratio, a 1:1 ratio, and a 1:5 ratio. The treatment plan involves cycling a patient through a set of formulations and selecting each subsequent formulation based on the patient's or patient family's feedback. The Halo BASE formulations are recommended by HCP for patients suffering from autism. The TF Formulations in Halo BASE include a variety of formulations that include cannabinoids and terpenes without the THC. Conditions like autism are very individualized. Therefore, the treatment must be individualized for each patient. The following Halo BASE formulations include ingredients and ratios that have shown to help patients with autism: a 20:1 ratio, a 1:1 ratio, and a 1:520 ratio. Halo D4 is a 1:20 CBD:THC ratio. Halo D2 is a 1:1 (CBD:THC ratio) plus 5 mgs of CBD. A more potent version of Halo D1, which is a 1:1 ratio, plus 5 mgs of CBG. Halo D3 is a 10:1 CBD:THC ratio. Halo D4 is a more potent version of D3, plus 10 mgs of CBG and 5 mgs of CBN. Halo D5 is a 1:5 CBD:THC ratio. Halo D6 is a more potent version of Variation D5, plus 5 mgs of CBG and 2 mgs of CBN. Studies have shown that patients with autism experience a significant reduction in symptoms.

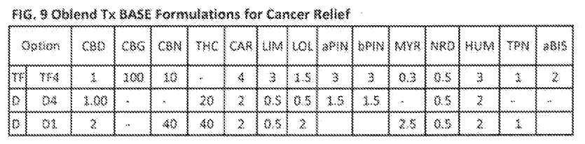

FIG. 9 Table shows Oblend® Tx BASE Formulations called “Armstrong.” Studies show that the Oblend Tx, Armstrong Base formulations provide relief to subjects suffering from cancer and cancer treatment-related symptoms including loss of appetite, sleep issues, nausea, vomiting, depression, and anxiety. Patients report improved sleep, increased appetite, and a decrease in pain and anxiety associated with cancer. Studies establish that there are two Armstrong daytime formulations: (1) Armstrong AM, a THC free formulation that should be taken three (3) times per day, and (2) Armstrong AM+, which includes THC. Armstrong AM+ is a supplement to Armstrong AM and should be taken as a supplement to Armstrong AM when the patient tolerates THC. For example, if a patient cannot tolerate THC while working or driving, then the patient would only take Armstrong AM. However, during the weekend, if the patient is not working or driving, then the patient may be able to tolerate the AM+ formulation with THC. In this case, the patient should take Armstrong AM and Armstrong AM+ to maximize the benefits of THC. The dosage amount of Armstrong AM+ can be titrated over time as the patient's body builds tolerance (habituation) to THC. Armstrong PM includes significant amounts of CBN, THC, MYR, LOL, and TPN but may make the patient sleepy if taken during the day. Armstrong BASE formulations also include an effective mixture of terpenes including αPIN, CAR, HUM, LEVI, LOL, and NRD.

The Armstrong Oblend Tx BASE formulations are helpful for subjects suffering from symptoms of cancer and cancer treatments, including loss of appetite, sleep issues, loss of appetite, nausea, vomiting, depression, and anxiety. These formulations include ingredients with cancer fighting properties, some of which kill certain types of cancer cells. THC, CBD, CBG and CBN slow growth and/or cause apoptosis in certain types of cancer cells. Certain cannabinoids slow growth and reduce spread of some forms of cancer. For example, THC is effective at reducing nausea and vomiting resulting from chemotherapy. THC may impact tumor cells and growth of cancerous cells. CBD reduces the symptoms of cancer and cancer treatments, such as nausea, vomiting, and depression. CBG has a cell proliferation characteristic in several cancer cell lines, including breast, prostate, and colorectal carcinoma, gastric adenocarcinoma, glioma, leukemia, and transformed thyroid cells. CBN is used to inhibit the growth of cancer cells. It is particularly effective in preventing the growth of breast cancer. The Armstrong BASE also includes terpenes with cancer fighting properties including limonene (toxic to breast cancer cell lines), alpha bisabol (induces apoptosis in models of leukemia), humulene (toxic to several cancer cell lines, including breast, leukemia, cervical, colorectal, and lung cells), alpha and beta pinene, beta caryophyllene, linalool, nerolidol, terpinolene also have anti-cancer properties.

There are two Armstrong daytime formulations: (a) Armstrong am, a THC free formulation that should be taken three (3) times per day, (2) Armstrong am +, which includes THC. Armstrong am + is a supplement to Armstrong am and is taken as a supplement to Armstrong am when the patient can tolerate THC. For example, if patient cannot tolerate THC while working or driving, then the patient would only take Armstrong am; however, during the weekend, if the patient is not working or driving, then the patient may be able to tolerate a formulation with THC. In this case, the patient should take Armstrong am and Armstrong am + to maximize the benefits of THC. The dosage amount of Armstrong am + can be titrated over time as the patient's body builds tolerance to THC. Armstrong pm includes significant amounts of CBN, THC, myrcene, linalool, and terpinolene which have proven anti-cancer properties but may make the patient sleepy if taken during the day.

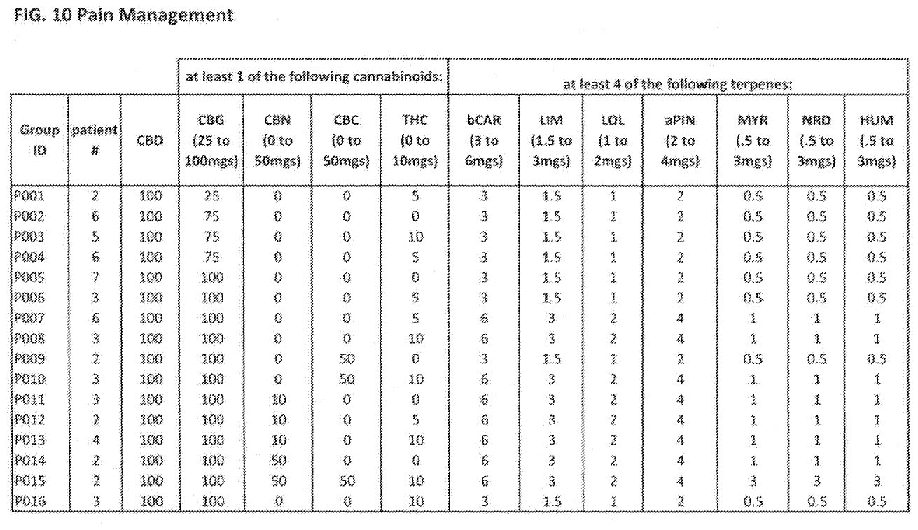

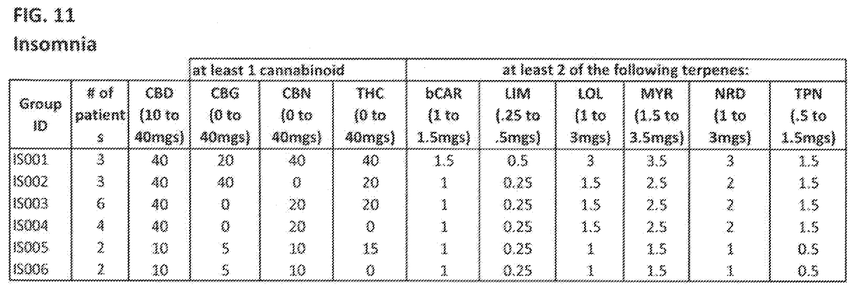

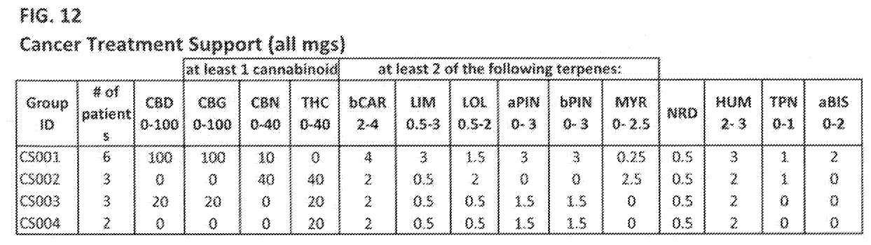

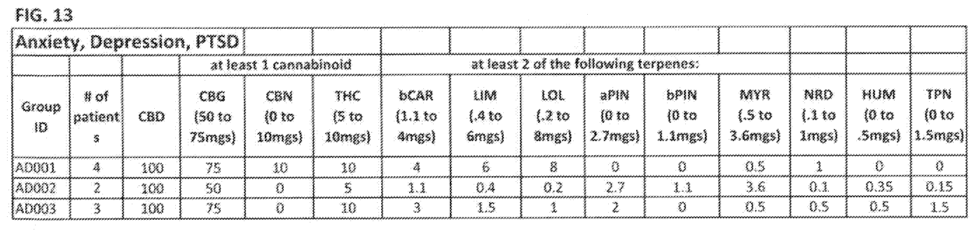

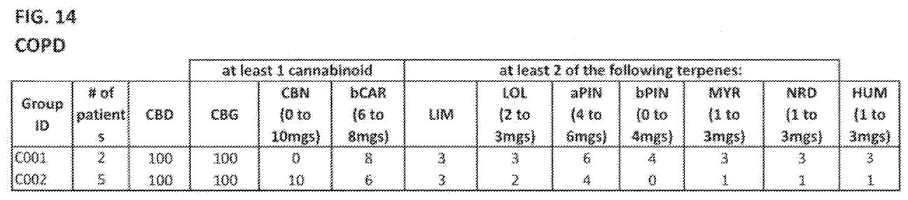

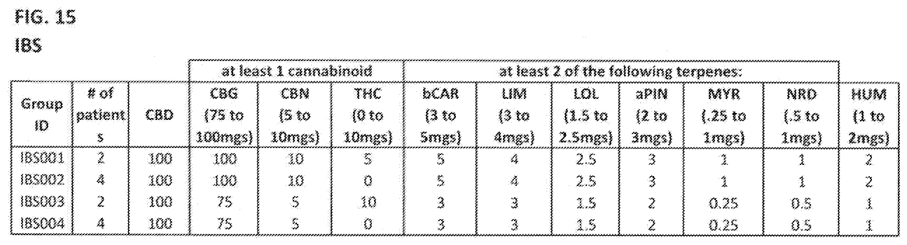

FIG. 10 is a Pain Management Table, FIG. 11 is an Insomnia Table, FIG. 12 is a Cancer Treatment Support Table, FIG. 13 is an Anxiety, Depression, PTSD Management Table, FIG. 14 is a Chronic Obstructive Pulmonary Disease (COPD) Table, and FIG. 15 is a Irritable Bowel Syndrome Table. These are symptom-specific tables which summarize the pharmaceutical compositions where two or more patients, grouped by specific composition have reported moderate to substantial symptom improvement with the use of the specified composition for periods of use ranging from 1 to 24 months. With respect to the Tables in FIGS. 10-15, the amounts specified in the tables represent the mass in milligrams (mg) per 1.0 milliliter (mL). Dosing protocols vary and may involve a dosing volume ranging from 0.1 ml to 1 ml with a frequency of once, twice or three times a day (QD, BID, or TID, respectively), and in some cases used on an as-needed basis.

Delta-8 THC was used in place of Delta-9 THC for these studies. Delta-8 is a naturally occurring isomer of Delta-9. Studies have found that Delta-8 to be a highly effective analog to Delta-9. The difference, according to most patients, is that Delta-8 is noticeably milder than Delta-9 and as a result, fewer side-effects generally associated with THC are reported when compared to compositions prepared with Delta-9. Given the relatively low dose utilized, reported side-effects may include a mild lethargy, anxiety, or a mild lack of mental clarity.

FIG. 10, Pain Management Table, summarizes the formulation variants for which groups of two or more patients have experienced moderate to substantial improvement with the severity and frequency of pain and inflammation. Data shows that in addition to those compositions associated with two or more successful “Pain” patient outcomes, the following pharmaceutical compositions associated with at least one successful outcome to date: for Migraine Management, a cannabinoid profile comprising: (a) CBD and CBG present at a ratio of 2:1 (wt/wt) where the CBD was included at a concentration of 133 mg/mL, and (b) a terpene profile comprising 4.5 mg/mL CAR, 2.25 mg/mL LEVI, 1.5 mg/mL LOL, 3.0 mg/mL αPIN, 0.75 mg/mL MYR, 0.75 mg/mL NRD, and 0.75 mg/mL HUM.

FIG. 11, Insomnia Table, summarizes the compositions for which groups of two or more patients experienced moderate to substantial improvement in time to sleep onset, sleep duration, and/or general sleep quality. Data shows that in addition to those compositions associated with two or more successful “Insomnia” patient outcomes, the following pharmaceutical compositions associated with one successful outcome to date: a cannabinoid profile comprising: (a) CBD and CBG present at a ratio of 1:1 (wt/wt) where the CBD was included at a concentration of 60 mg/mL, and (b) a terpene profile comprising 1.0 mg/mL CAR, 0.25 mg/mL LEVI, 1.5 mg/mL LOL, 2.50 mg/mL MYR, 2.0 mg/mL NRD and 1.5 mg/mL TPN.

FIG. 12, Cancer Treatment Support Table, summarizes those compositions for which groups of two or more patients experienced moderate to substantial relief from the side effects of chemo and radiation therapy and disease related symptoms including pain, nausea, anxiety, depression, and loss of appetite. Patients using Armstrong BASE formulations have reported improved sleep, an increase in appetite, and a decrease in nausea, pain, and anxiety associated with cancer treatment.

FIG. 13 is an Anxiety, Depression, PTSD Management Tables, summarize the compositions for which groups of two or more patients experienced moderate to substantial improvement with symptoms related to these often-overlapping mental conditions including racing thoughts, panic attacks, loss of motivation and joy related to things that are normally pleasurable, night terrors, and loss of sleep. Cannabinoids consistent for all anxiety and PTSD patients but the terpene profiles are adjusted. Some people with anxiety respond well to limonene; however, limonene make some people more anxious.

FIG. 14, Chronic Obstructive Pulmonary Disease (COPD) Table, shows relief formulations. In addition to those compositions associated with two or more successful COPD patient outcomes, the following pharmaceutical compositions associated with one successful outcome to date: A cannabinoid profile comprising: (a) CBD, CBG, and D8 THC present at a ratio of 1:1 (wt/wt) CBD to CBG and a ratio of 10:1 (wt/wt) CBD to D8 THC where CBD was included at a concentration of 100 mg/mL, and (b) a terpene profile comprising 6.0 mg/mL CAR, 3.0 mg/mL LEVI, 3.0 mg/mL LOL, 4.0 mg/mL αPIN, 1.0 mg/mL MYR, 1.0 mg/mL NRD, and 1.0 mg/mL TPN.

FIG. 15, Irritable Bowel Syndrome Table, shows relief formulations. In addition to those compositions associated with two or more successful IBS patient outcomes, the following pharmaceutical compositions associated with one successful outcome to date: a cannabinoid profile comprising: (a) CBD, CBG, D8 THC, and CBC present at a ratio of 1:1 (wt/wt) CBD to CBG, 10:1 CBD to D8 THC, and 2:1 (wt/wt) CBD to CBC where CBD was included at a concentration of 100 mg/mL, and (b) a terpene profile comprising 4.5 mg/mL CAR, 4.5 mg/mL LEVI, 2.25 mg/mL LOL, 3.0 mg/mL αPIN, 0.375 mg/mL MYR, 0.75 mg/mL NRD, and 1.5 mg/mL TPN

According to one implementation, patient feedback guides the HCP to adjust the initial BASE formulations. This may include multiple patient—HCP interactions, including visits, phone calls, emails, or an app, to allow for and document patient feedback. Based on the patient's feedback, BASE formulations ingredients are adjusted, if needed. Patient follow up can be handled by the HCP or another entity, based on provider's preference.

According to one implementation, a consultation and training Delivery Platform (“DP”) Assist (which includes the E.H.R. input forms) provides the HCP, with further consultation forms, provides monthly provider reports via the DP platform, detailed patient reports and/or efficacious data on products, formulations, patient demographics and more. The DP Assist can also include continual HC staff training (if desired) of the endocannabinoid system, information on traditional pharma vs botanical medicine, and/or medicinal properties of cannabinoids and terpenoids.

As an illustrative example, customized formulations in line with the present disclosure can include dosing instructions. For example, dosing instructions can include informing a patient that: tincture bottles have droppers sized to deliver 0-1.00 mL, calibrated in 0.25 mL increments. (a) Start with 0.25 mL, 2-3 times per day for daytime formulations or mL, 45 minutes before bed for night time formulations. (b) Place drops under tongue and hold under tongue for 90 seconds for best effect. (c) Repeat for 5 days. Consistency is important as the product will rebalance the patient's endocannabinoid system over that time. Taking these oil drops only when patient feels the need may help but such “as needed” or PRN application will not result in an optimal outcome. (d) After 5 days, if patient is not experiencing the relief the care giver had hoped for, increase the patient's dose by 0.25 mL or as recommend by the HC provider.

Further, the customization process of the present disclosure can involve patient input regarding whether an initial product alleviates all or any of a patient's symptoms. In this way, feedback can be provided by, for example, email or phone.

One study of the primary medical conditions for 322 total patients indicated: 71% sought relief for pain, 13% anxiety/PTSD, and 9% insomnia, making up 92% of all patients seeking relief from pain, anxiety, or insomnia. Following treatment, 74.5% of patients providing regular feedback reported positive outcomes. Within the illustrative example, the best and most consistent symptom relief came to those who had reported pain, anxiety, and PTSD. In this study, insomnia and sleep issues typically required more adjustments to a formulation, but also resulted in more positive outcomes. In the illustrative example, 33% of patients provided little or no feedback. Outcome comparisons for patients treated with DELTA 8 vs. THC-free formulations show 88% reporting positive treatment outcomes with DELTA 8 vs. 87% without. According to the studies, most patients have an optimized formulation with 1-2 adjustments. This study reveals that the initial formulation had a reasonable degree of efficacy and utility.

Depending on the patient's symptoms, the HCP start with one Oblend® BASE Treatment Plan based upon the patient's description of his or her condition. The treatment plans are derived from research and clinical trials of which ingredients are efficacious for the described medical condition(s). Some treatment plans involve changing the terpene profiles (i.e., anxiety—very personal . . . not one size fits all . . . all about the terpene profile). Others, like pain, keep the same terpene profile; however, the total terpene content is increased as the active ingredients increase to keep the terpene % within a level of the active ingredients (i.e., between 3-16%).

Also, some patients (about 25% of the patient population) start with one of basic treatment plan but, based on patient feedback, move into a custom formulation. Illustrative discoveries include: (A) Sleep: if a patient is struggling with longevity of sleep, then increase any of the following ingredients: terpinolene, myrcene, CBN, CBDV. (B) Pain: Add terpinolene to existing RELIEF BASE treatment plan, increase nerolidol, humulene and decrease the delta 8 THC. (C) Anxiety (this is on a sliding scale but it is important.) (D) Alpha pinene works well for COPD, plus maybe additional humulene, beta caryophyllene, nerolidol CBD CBG. (E) Alpha pinene and limonene is used for cognitive decline and/or neurodegenerative diseases. (F) Adjustments are made for the delta 8 THC based on each individual patient responses to formulation and how it affects patient personally. Some patients take more at night to sleep through the night. (F) Most formulations include nerolidol; however, studies have found that some patients are allergic to nerolidol. In such cases, a removal of that ingredient from the formulation and may or may not require an increase the other terpenes.

Studies have shown that the following Class A-E formulations are efficacious. The designation of Class A, B, etc. does not indicate that one class of formulations is better or worse than another, that is, the Class designation is only for differentiating the various formulations. For the treatment of pain, the following Class A formulations and methods show reasonable patient improvement.

A1. A method of treating pain, comprising, administering an effective amount of a liquid composition comprised of, on a per milliliter (ml) basis, about 20-133 mgs cannabidiol (CBD); up to about 125 mgs cannabigerol (CBG); in a range from 0 mgs to about 50 mgs cannabinol (CBN); in a range from 0 mgs to about 50 mgs cannabichromene (CBC); in a range from 0 mgs to about 50 mgs delta-8-tetrahydrocannabinol (delta 8 THC); and a nominal effective amount of at least two terpenes from the group of terpenes including: about 1.0-6.0 mgs beta caryophyllene; in a range from 0 mgs to about 4.5 mgs alpha Pinene; in a range from 0 mgs to about 1.5 mg beta Pinene; about 0 mgs and 3.0 mgs limonene; about 0.1-3.0 mgs linalool; about 0.5-3.6 mgs myrcene; about 0.5-3.0 mgs nerolidol; in a range from 0 mgs to about 3.0 mgs humulene; and in a range from 0 mgs to about 1.5 mgs terpinolene; to a patient suffering from pain.

A2. The method of treating pain as claimed in claim 1 (referring to A1 in this Class) wherein the composition includes an effective amount of delta-8-tetrahydrocannabinol (delta 8 THC) in a range from about 1.25 mg/ml to 50 mgs/ml. A3. The method of treating pain as claimed in claim 2 wherein the composition includes CBG from about 25 to about 125 mgs/ml. A4. The method of treating pain as claimed in claim 2 wherein the composition includes CBC from 10 to about 50 mgs/ml. A5. The method of treating pain as claimed in claim 1 wherein the composition includes delta 8 THC from about 1 mg/ml to about 50 mgs/ml.

A6. The method of treating pain as claimed in claim 1 wherein the composition includes CBN from about 5 to about 50 mgs/ml. A7. The method of treating pain as claimed in claim 2 wherein the composition includes alpha Pinene from about 1 mg/ml up to about 4.5 mgs/ml. A8. The method of treating pain as claimed in claim 2 wherein the composition includes beta Pinene from about 0.1 mg/ml to 3.0 mgs/ml. A9. The method of treating pain as claimed in claim 2 wherein the composition includes limonene from about 0.5 mg/ml to 3.0 mgs/ml. A10. The method of treating pain as claimed in claim 2 wherein the composition includes linalool from about 0.5 mg/ml to 3.0 mgs/ml.

A11. The method of treating pain as claimed in claim 2 wherein the composition includes myrcene from about 0.5 mg/ml to 3.6 mgs/ml. A12. The method of treating pain as claimed in claim 2 wherein the composition includes nerolidol from about 0.1 mg/ml to 3.0 mgs/ml. A13. The method of treating pain as claimed in claim 2 wherein the composition includes humulene from about 0.1 mg/ml to 3.0 mgs/ml. A14. The method of treating pain as claimed in claim 2 wherein the composition includes terpinolene from about 0.5 mg/ml to 1.5 mgs/ml.

A15. The method of treating pain as claimed in claim 1 wherein the daily dose of the composition comprises about 0.25 to 1.0 ml administered two or three times per day as needed. A16. The method of treating pain as claimed in claim 13 wherein the composition is applied underneath a tongue and the dose is held under the tongue for about at least 30 seconds. A17. The method of treating pain as claimed in claim 1 wherein the cannabidiol (CBD) is botanical CBD, and the two or more terpenes are a botanical caryophyllene; a botanical limonene; a botanical linalool; a botanical myrcene; and a botanical nerolidol. A18. The method of treating pain as claimed in claim 1 wherein the following components, if present in the composition, are botanical components: CBD, CBG, CBN, CBC, delta 8 THC, beta caryophyllene, alpha Pinene, beta Pinene, limonene, linalool, myrcene, nerolidol, humulene, and terpinolene.

For the treatment of insomnia and nighttime pain, the following Class B formulations and methods show reasonable patient improvement. Class B: Inventive Compositions for Insomnia and Nighttime Pain.

B1. A method of treating insomnia due to nighttime pain, comprising administering an effective amount of a liquid composition comprised of, on a per milliliter (ml) basis: about −108 mgs cannabidiol (CBD); 0-60 mgs cannabigerol (CBG); 0-40 mgs cannabinol (CBN); mgs delta-8-tetrahydrocannabinol (delta 8 THC); and a nominal effective amount of a terpene, from the group of terpenes, including about 1.5-3.5 mgs myrcene; about 0.5-3.0 mgs nerolidol; about 0.25-1.5 mgs beta caryophyllene; about 0-1.5 mgs terpinolene; about 1.0-3.0 linalool; and about 0.25-0.50 mgs limonene, to a patient suffering from insomnia.

B2. The method of treating insomnia as claimed in claim 1 (referring to B1 in this Class) wherein the composition includes about 5-108 mgs CBD. B3. The method of treating insomnia as claimed in claim 1 wherein the composition includes CBG up to about 60 mgs of CBG. B4. The method of treating insomnia as claimed in claim 1 wherein the composition includes CBN up to about 40 mgs. B5. The method of treating insomnia as claimed in claim 1 wherein the composition includes delta 8 THC up to about 40 mgs delta 8 THC.

B6. The method of treating insomnia as claimed in claim 1 wherein the composition includes about 1.5-3.5 mgs of myrcene. B7. The method of treating insomnia as claimed in claim 1 wherein the composition includes about 0.5-3.0 mgs of nerolidol. B8. The method of treating insomnia as claimed in claim 1 wherein the composition includes about 0.25-1.5 mgs of beta caryophyllene. B9. The method of treating insomnia as claimed in claim 1 wherein the composition includes about 0-1.5 mgs of terpinolene. B10. The method of treating insomnia as claimed in claim 1 wherein the composition includes about 0.25-0.5 mgs of limonene.

B11. The method of treating insomnia as claimed in claim 1 wherein the composition includes about 1-3 mgs of linalool. B12. The method of treating insomnia as claimed in claim 1 wherein the daily dose of the composition comprises about 0.25 to 1.0 ml administered one time per day. B13. The method of treating insomnia as claimed in claim 1 wherein the composition is applied underneath a tongue and the dose is held under the tongue for at least 30 seconds. B14. The method of treating insomnia as claimed in claim 1 wherein the cannabidiol (CBD) is botanical CBD, and the two or more terpenes are a botanical beta caryophyllene; a botanical limonene; a botanical linalool; a botanical myrcene; a botanical terpinolene; and a botanical nerolidol. B15. The method of treating insomnia as claimed in claim 1 wherein the following components, if present in the composition, are botanical components: CBD, CBG, CBN, delta 8 THC, beta caryophyllene, alpha Pinene, beta Pinene, limonene, linalool, myrcene, nerolidol, and terpinolene.

For the treatment of ANXIETY #1 (CALM), the following Class C formulations and methods show reasonable patient improvement. Class C: Inventive Compositions for Anxiety and Depression. These often-overlapping symptoms involve a state of worry or mental tension or persistent sadness and a lack of interest or pleasure in previously rewarding or enjoyable activities.

C1. A method of treating anxiety and post-traumatic stress disease (PTSD), comprising, administering an effective amount of a liquid composition comprised of, on a per milliliter (ml) basis, about 2.5-100 mgs cannabidiol (CBD); and at least one of (i) about 0-100 mgs cannabigerol (CBG); (ii) 0-100 mgs cannabinol (CBN); and (iii) 0-40 mgs delta-8-tetrahydrocannabinol (delta 8 THC); and the liquid composition includes an effective amount of at least one terpene from the group of terpenes including about 0.5-4.0 mgs beta caryophyllene; about 0.75-6.0 mgs limonene; about 1.0-8.0 mgs linalool; about 0.0625-mgs myrcene; and about 0.5-1.0 mgs nerolidol, to a patient suffering from anxiety.

C2. The method of treating anxiety and PTSD as claimed in claim 1 (referring to treatment C1 in this Class) wherein the composition includes CBG up to about 100 mgs. C3. The method of treating anxiety and PTSD as claimed in claim 1 wherein the composition includes CBN up to about 100 mgs. C4. The method of treating anxiety and PTSD as claimed in claim 1 wherein the composition includes Delta 8 THC up to about 40 mgs. C5. The method of treating anxiety and PTSD as claimed in claim 1 wherein the composition includes beta caryophyllene up to about 4.0 mgs.

C6. The method of treating anxiety and PTSD as claimed in claim 1 wherein the composition includes limonene up to about 6.0 mgs. C7. The method of treating anxiety and PTSD as claimed in claim 1 wherein the composition includes limonene up to about 8.0 mgs. C8. The method of treating anxiety and PTSD as claimed in claim 1 wherein the composition includes myrcene up to about 0.5 mgs. C9. The method of treating anxiety and PTSD as claimed in claim 1 wherein the composition includes nerolidol up to about 1.0 mgs.

For the treatment of ANXIETY #2, the following Class D formulations and methods show reasonable patient improvement. Class D: Inventive Compositions for Anxiety #2 (Serenity).

D1. A method of treating anxiety (SERENITY), comprising, administering an effective amount of a liquid composition comprised of, on a per milliliter (ml) basis: 2.5-100 mgs cannabidiol (CBD); and a nominal effective amount of at least one of a 0-100 mgs cannabigerol (CBG); 0-100 mgs cannabinol (CBN); 0-40 mgs delta-8-tetrahydrocannabinol (delta 8 THC); and an effective amount of at least one terpene from the group of terpenes including 0.275-2.2 mgs beta caryophyllene; 0.1-0.8 mgs limonene; 0.05-0.4 linalool; 0.675-5.41 alpha pinene; 0.275-2.2 beta pinene; 0.9-9.0 myrcene; 0.025-0.25 nerolidol; 0.0875-0.7 humulene; 0.0375-0.3 terpinolene; and 0-8.0 mgs linalool, to a patient suffering from anxiety.

D2. The method of treating anxiety (SERENITY) as claimed in claim 1 (referring to D1 in this treatment Class) wherein the composition includes about 10.0-100 mgs of CBD. D3. The method of treating anxiety (SERENITY) as claimed in claim 1 wherein the composition includes CBG up to about 100 mgs. D4. The method of treating anxiety (SERENITY) as claimed in claim 1 wherein the composition includes CBN in an amount up to about 100 mgs. D5. The method of treating anxiety (SERENITY) as claimed in claim 1 wherein the composition includes Delta 8 THC in an amount up to about 40 mgs. D6. The method of treating anxiety (SERENITY) as claimed in claim 1 wherein the composition includes about 0.275-2.2 mgs of beta caryophyllene. D7. The method of treating anxiety (SERENITY) as claimed in claim 1 wherein the composition includes about 0.1-0.8 mgs of limonene. D8. The method of treating anxiety (SERENITY) as claimed in claim 1 wherein the composition includes about 0.05-0.4 mgs of linalool. D9. The method of treating anxiety (SERENITY) as claimed in claim 1 wherein the composition includes about 0.675-5.4 mgs of alpha pinene. D10. The method of treating anxiety (SERENITY) as claimed in claim 1 wherein the composition includes about 0.275-2.2 mgs of beta pinene.

D11. The method of treating anxiety (SERENITY) as claimed in claim 1 wherein the composition includes about 0.9-7.2 mgs of myrcene. D12. The method of treating anxiety (SERENITY) as claimed in claim 1 wherein the composition includes about 0.025-0.2 mgs. Of nerolidol. D13. The method of treating anxiety (SERENITY) as claimed in claim 1 wherein the composition includes about 0.0875-0.7 mgs of humulene. D14. The method of treating anxiety (SERENITY) as claimed in claim 1 wherein the composition includes about 0.0375-mgs of terpinolene.

For the treatment of irritable bowel syndrome (IBS), the following Class E formulations and methods show reasonable patient improvement. Class E: Inventive Compositions for Irritable Bowel Syndrome.

E1. A method of treating Irritable Bowel Syndrome (IBS) comprising, administering an effective amount of a liquid composition comprised of, on a per milliliter (ml) basis: 100 mgs cannabidiol (CBD); and at least one of (i) 0-100 mgs cannabigerol (CBG); (ii) 0-20 mgs cannabinol (CBN); and (iii) 0-50 mgs delta-8-tetrahydrocannabinol (delta 8 THC); and the composition includes an effective amount of at least one terpene from the group of terpenes including 3.0-5.0 mgs beta caryophyllene; 2.0-3.0 mgs alpha pinene; 3.0-5.0 mgs limonene; 1.5-2.5 mgs linalool; 0.25 to 1.0 mgs myrcene; 0.50-1.0 mgs nerolidol; and 1 to 2 mgs humulene to a patient suffering from IBS.

E2. The method of treating IBS as claimed in claim 1 (referring to treatment E1 in this Class) wherein the composition includes CBG in an amount up to about 100 mgs. E3. The method of treating IBS as claimed in claim 1 wherein the composition includes CBN in an amount up to about 100 mgs. E4. The method of treating IBS as claimed in claim 1 wherein the composition includes about 3.0-5.0 mgs beta caryophyllene. E5. The method of treating IBS as claimed in claim 1 wherein the composition includes about 3.0-5.0 mgs of limonene. E6. The method of treating IBS as claimed in claim 1 wherein the composition includes about 1.5-2.5 mgs linalool. E7. The method of treating IBS as claimed in claim 1 wherein the composition includes about 2.0-3.0 mgs alpha pinene. E8. The method of treating IBS as claimed in claim 1 wherein the composition includes about 0.25 to 1.0 mgs myrcene. E9. The method of treating IBS as claimed in claim 1 wherein the composition includes about 0.50-1.0 mgs nerolidol. E10. The method of treating IBS as claimed in claim 1 wherein the composition includes about 1.0-2.0 mgs of humulene.

The claims appended hereto are meant to cover the scope and spirit of the present inventive compositions and treatments.

1. A method of treating pain, comprising, administering an effective amount of a liquid composition comprised of, on a per milliliter (ml) basis, about 10-120 mgs cannabidiol (CBD); up to about 100 mgs cannabigerol (CBG); in a range from 0 mgs to about 50 mgs cannabinol (CBN); in a range from 0 mgs to about 50 mgs cannabichromene (CBC); in a range from 0 mgs to about 50 mgs delta-8-tetrahydrocannabinol (delta 8 THC); and a nominal effective amount of at least two terpenes from the group of terpenes including: about 0.25-6.0 mgs beta caryophyllene; in a range from 0 mgs to about 4.5 mgs alpha Pinene; in a range from 0 mgs to about 1.5 mg beta Pinene; about 0.6-3.0 mgs limonene; about 0.1-3.0 mgs linalool; about 0.5-3.6 mgs myrcene; about 0.5-3.0 mgs nerolidol; in a range from 0 mgs to about 3.0 mgs humulene; and in a range from 0 mgs to about 1.5 mgs terpinolene; to a patient suffering from pain.

2. The method of treating pain as claimed in claim 1 wherein the composition includes an effective amount of delta-8-tetrahydrocannabinol (delta 8 THC) in a range from about 1.25 mg/ml to 50 mgs/ml.

3. The method of treating pain as claimed in claim 2 wherein the composition includes CBG up to about 100 mgs/ml.

4. The method of treating pain as claimed in claim 2 wherein the composition includes CBC up to about 50 mgs/ml.

5. The method of treating pain as claimed in claim 1 wherein the composition includes delta 8 THC up to about 50 mgs/ml.

6. The method of treating pain as claimed in claim 1 wherein the composition includes CBN up to about 50 mgs/ml.

7. The method of treating pain as claimed in claim 2 wherein the composition includes alpha Pinene up to about 4.5 mgs/ml.

8. The method of treating pain as claimed in claim 2 wherein the composition includes limonene from about 0.1 mg/ml to 3.0 mgs/ml.

9. The method of treating pain as claimed in claim 2 wherein the composition includes linalool from about 0.1 mg/ml to 3.0 mgs/ml.

10. The method of treating pain as claimed in claim 2 wherein the composition includes myrcene from about 0.5 mg/ml to 3.6 mgs/ml.

11. The method of treating pain as claimed in claim 2 wherein the composition includes nerolidol from about 0.5 mg/ml to 3.0 mgs/ml.

12. The method of treating pain as claimed in claim 2 wherein the composition includes terpinolene from about 0 mg/ml to 1.5 mgs/ml.

13. The method of treating pain as claimed in claim 1 wherein the daily dose of the composition comprises about 0.25 to 1.0 ml administered two or three times per day as needed.

14. The method of treating pain as claimed in claim 13 wherein the composition is applied underneath a tongue and the dose is held under the tongue for about at least 30 seconds.

15. The method of treating pain as claimed in claim 1 wherein the cannabidiol (CBD) is botanical CBD, and the two or more terpenes are a botanical caryophyllene; a botanical limonene; a botanical linalool; a botanical myrcene; and a botanical nerolidol.

16. The method of treating pain as claimed in claim 1 wherein the following components, if present in the composition, are botanical components: CBD, CBG, CBN, CBC, delta 8 THC, beta caryophyllene, alpha Pinene, beta Pinene, limonene, linalool, myrcene, nerolidol, humulene, and terpinolene.
